(19) United States
(12) Patent Application Publication
(10) Pub. No.: US 2006/0251631 A1
(43) Pub. Date: Nov. 9, 2006

(54) TREATMENT OF JOINT DISEASE, METHODS AND APPARATUSES THEREFOR

(75) Inventors: H. Davis Adkisson IV, St. Louis, MO (US); Mitchell S. Seyedin, Monte Sereno, CA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: ISTO Technologies, Inc., St. Louis, MO (US)

(21) Appl. No.: 11/380,308

(22) Filed: Apr. 26, 2006

Related U.S. Application Data
(60) Provisional application No. 60/678,087, filed on May 5, 2005.

(51) Int. Cl. A61K 48/00; C12N 5/08; A61K 38/39; A61K 31/722; A61K 31/728

(52) U.S. Cl. 424/93.21; 435/366; 514/12; 514/54; 514/55

(57) ABSTRACT

The present application discloses compositions, methods and devices for treatment of degenerative cartilaginous structures of an arthritic joint, including articular cartilage and the meniscus. A composition can comprise chondrocytes expressing type II collagen and a biological macromolecule such as hyaluronic acid or a collagen. The chondrocytes can be obtained from hyaline cartilage of human cadavers up to about two weeks following death, and can be grown in vitro. A composition can be delivered to a recipient by intra-articular injection. Examples of joints into which a composition can be injected include a knee joint, a hip joint, a shoulder joint, an ankle joint, a wrist joint, a digit joint and an elbow joint.

### RELATED APPLICATION

This application claims the benefit of priority of U.S. provisional application Ser. No. 60/678,087 filed May 5, 2005, which application is incorporated herein by reference in its entirety.

### BACKGROUND

Joint disease is a leading cause of pain and disability in the adult population. For many individuals, a joint disease such as osteoarthritis can become a chronic affliction. The morbidity associated with joint disease and its spectrum of associated disorders is responsible for significant health care, economic and social costs. Current treatments for repairing or ameliorating joint disease can be expensive, poorly effective, painful, or lengthy. Alternative treatments are, therefore, needed.

### SUMMARY

In view of the need for treatments for joint diseases such as osteoarthritis, the present inventors have devised compositions, methods and devices for repair, replacement and/or supplementation of a joint which involve injection of hyaline chondrocytes into a diseased joint.

Accordingly, the present teachings disclose, in certain embodiments of the invention, methods of treating joint disease such as osteoarthritis in a mammal in need thereof. In these embodiments, a method comprises forming a composition comprising chondrocytes expressing type II collagen and at least one biological macromolecule; and injecting the composition into a diseased joint in the mammal.

In other embodiments, the present teachings disclose an apparatus configured for injection of chondrocytes expressing type II collagen into a diseased non-intervertebral joint of a mammal. In these embodiments, the apparatus comprises a reservoir, wherein the reservoir holds therewithin a composition comprising at least one biological macromolecule and chondrocytes expressing type II collagen, and at least one hollow tube which inserts into the diseased non-intervertebral joint, wherein the hollow tube communicably connects with the reservoir.

### BRIEF DESCRIPTION OF THE DRAWINGS

These and other features, aspects, and advantages of the present invention will become better understood with regard to the following description, appended claims and accompanying figures where:

FIG. 1 illustrates lack of chondrocyte alloreactivity in mixed co-cultures of chondrocytes and lymphocytes.

FIG. 2 illustrates chondrocyte inhibition of active T lymphocyte proliferation.

FIG. 3 illustrates that chondrocyte-mediated immunosuppression of activated T cells requires cell-to-cell contact.

FIG. 4 illustrates flow cytometric staining profiles of CD11c, MHC II, CD80 and CD86 cell surface markers for bone marrow derived dendritic cells.

FIG. 5 illustrates flow cytometric staining profiles of CD11c, MHC II, CD80 and CD86 cell surface markers for chondrocytes obtained from two different donors.

FIG. 6 illustrates expression profiles for selected genes in a chondrosarcoma cell line and chondrocytes from donors of different ages.

### DETAILED DESCRIPTION

The present teachings include compositions, methods and devices for repair, replacement and/or supplementation of a diseased joint. These methods involve injection of chondrocytes into a diseased joint such as an osteoarthritic joint.

The methods and compositions described herein utilize laboratory techniques well known to skilled artisans and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, NY, 1998; and Harlow, E., Using Antibodies: A Laboratory Manual, Cold Spring Harbor Laboratory Press, Cold Spring Harbor, N.Y., 1999.

In various embodiments, the present teachings include methods of repairing a diseased joint in a mammal in need of treatment, such as a human patient suffering from osteoarthritis. In various aspects, the osteoarthritis can comprise a degenerative articular cartilage, and/or a degenerative meniscus. In some aspects, the diseased joint can be any joint other than an intervertebral disc. In non-limiting example, a diseased joint which can be subject to repair in accordance with the present teachings can be a knee joint, a hip joint, a shoulder joint, an ankle joint, a wrist joint, a digit joint or an elbow joint. In non-limiting example, a composition of the present teachings can be injected directly into the synovial cavity of a knee joint.

Accordingly, the present teachings disclose methods of treating joint disease in a mammal in need thereof, in which a method comprises forming a composition comprising chondrocytes expressing type II collagen and at least one biological macromolecule, and injecting the composition into a diseased joint in the mammal. Methods of these embodiments can further comprise growing the chondrocytes expressing type II collagen in vitro, for example as described in Adkisson, H. D., et al., Clin. Orthop. 391S, S280-S294, 2001; and U.S. Pat. Nos. 6,235,316 and 6,645,316 to Adkisson.

The chondrocytes of these embodiments can be human hyaline chondrocytes, and can be chondrocytes which express not only type II collagen, but also express high molecular weight sulfated proteoglycan.

In various configurations of the embodiments of the present teachings, the chondrocytes expressing type II collagen can be cadaver chondrocytes expressing type II collagen. As used herein, the term “cadaver chondrocytes” refers to viable chondrocytes originally comprised by a human cadaver, as well as clonal descendants of such chondrocytes, such as chondrocytes grown in vitro. Cadaver chondrocytes for use in the various aspects of the present teachings can be obtained from a human cadaver from tissues comprising chondrocytes, such as cartilage tissue. Such tissues can be dissected from a cadaver using standard dissection methods well known to skilled artisans. The chondrocytes utilized in the present teachings are hyaline cartilage chondrocytes, such as, for example, chondrocytes originating in hyaline cartilage of trachea, larynx, articular cartilage, or a combination thereof. Viable chondrocytes can be chondrocytes obtained from cartilaginous tissues in a donor cadaver for up to about two weeks after death of the donor. Accordingly, in some configurations, the time interval from the time of death of a donor (as determined, for example, by a physician or a coroner) to the time of dissection of cartilage tissue for isolation of chondrocytes from the donor can be any time following a pronouncement of death, up to about two weeks following death, such as, without limitation, about one hour, about one day, about two days, about three days, about four days, about five days, about six days, about seven days, about eight days, about nine days about ten days, about eleven days, about twelve days, about thirteen days, or about fourteen days after death. The term “isolation of chondrocytes” (and similar terms), as used herein, refers to separation of chondrocytes from a donor body or cadaver so as to yield a collection of chondrocytes that is substantially free of other cell types. In addition, a donor cadaver can be of any chronological age at time of death. For example, a donor cadaver can be, at time of death, post-natal, ten years old or younger, or fourteen years old or younger. A donor cadaver need not be a familial member of a recipient, or be otherwise matched immunologically with the recipient. Without being limited by theory, it is believed that the chondrocytes expressing type II collagen comprise an “immunologically privileged” cell type, so that such chondrocytes injected to a living recipient such as a human patient are not subject to rejection by the recipient's immune system. The immune-privileged status of the chondrocytes of the present teachings stand in contrast to, for example, allogeneic chondrocyte-enriched cultures derived from bone marrow, which evoke in a recipient immune responses such as fibrosis or progressive joint arthrosis (Butnariu-Ephrat, M., et al., Clinical Orthopaedics and Related Research 330, 234-243, 1996).

Cartilage tissue can be removed from a cadaver using any surgical or dissecting techniques and tools known to skilled artisans. Following cartilage removal from a cadaver, the cartilage tissue can be minced, dissociated into single cells or small groups of cells, and/or placed into tissue or cell culture using standard techniques and apparatuses well known to skilled artisans, such as techniques and apparatuses described in the these references. Non- limiting descriptions of methods of cartilage and chondrocyte removal and culture can be found in references such as, for example, Feder, J. et al. in: Tissue Engineering in Musculoskeletal Clinical Practice. American Academy of Orthopaedic Surgeons, 2004; Adkisson, H.D. et al., Clin. Orthop. 391S, S280-S294, 2001; and U.S. Pat. Nos. 6,235,316 and 6,645,316 to Adkisson.

Cadaver chondrocytes used in the various embodiments of the present teachings are all cadaver chondrocytes which express type II collagen, and in some configurations can be chondrocytes expressing other molecular markers such as a high molecular weight sulfated proteoglycan, such as, for example, aggrecan or chondroitin sulfate (Kato, Y., and Gospodarowicz, D., J. Cell Biol. 100: 477-485. 1985). The presence of such markers can be determined using materials and methods well known to skilled artisans, such as, for example, antibody detection and histological staining.

In various aspects of these embodiments, a biological macromolecule which can be comprised by a composition of the present teachings can be, in non-limiting example, hyaluronic acid, type I collagen, type III collagen, fibrinogen, fibrin, thrombin, pectin, chitosan, or a combination thereof. In some aspects, hyaluronic acid comprised by a composition can be high molecular weight hyaluronic acid, i.e., hyaluronic acid having an average molecular mass of about 1×10⁶ daltons, or greater. High molecular weight hyaluronic acid can be extracted or purified from biological sources using methods known to skilled artisans, for example methods disclosed in references such as McGary, C. T., et al., Methods in Enzymology 363, 354-365, 2003. High molecular weight hyaluronic acid also can be obtained from commercial sources, such as Bio-Technology General Ltd., Rehovot, Israel or Hyaluron, Inc., Burlington, Mass. The molecular mass of the hyaluronic acid can be determined by any method known to skilled artisans, such as, for example, by methods disclosed in Hokputsa, S., et al., European Biophysical J. 32, 450-456, 2003.

In certain alternative aspects, a biological macromolecule comprised by a composition can be, instead of or in addition to high molecular weight hyaluronic acid, a collagen such as type I collagen, type III collagen, or a combination thereof.

In other embodiments of the present teachings, the present inventors contemplate an apparatus configured for injection of chondrocytes expressing type II collagen into a diseased non-intervertebral joint of a mammal. In these embodiments, an apparatus can comprise a reservoir comprising a composition comprising at least one biological macromolecule such as high molecular weight hyaluronic acid as described above and chondrocytes expressing type II collagen, and at least one hollow tube which inserts into the diseased joint. In various configurations, the hollow tube can be communicably connected with the reservoir, and thereby provide a conduit for transferring the composition from the reservoir to a diseased joint. In various aspects, the hollow tube can be a hollow needle. In these configurations, the chondrocytes expressing type II collagen can be human chondrocytes, such as the chondrocytes described above, and can be obtained as described above. In addition, an apparatus of these embodiments can be configured for injection of chondrocytes into a diseased non-intervertebral joint of a mammal such as, in non-limiting example, a knee joint, a hip joint, a shoulder joint, an ankle joint, a wrist joint, a digit joint or an elbow joint.

The term “reservoir,” as used herein, refers to a part of an apparatus in which is held a fluid mixture, such as a composition of the present teachings.

In various configurations, a composition described herein can be placed into an apparatus or device configured for injection of chondrocytes into ajoint of a patient suffering from a joint disease such as osteoarthritis. Non-limiting examples of apparatuses and devices which can be configured for injection of chondrocytes into a joint include a biopsy instrument or transplantation instrument comprising a hollow tube or needle, a syringe, a double syringe, a hollow tube, a hollow needle such as a Jamshidi needle, a Cook needle (Cook incorporated, Bloomington, Ind. USA), a cannula, a catheter, a trocar, a stylet, an obturator, or other instruments, needles or probes for cell or tissue injection known to skilled artisans. Furthermore, surgical techniques for injecting a composition comprising chondrocytes and a biological macromolecule as described herein can be adapted from well-established techniques for introduction of a fluid into a degenerative joint of a patient.

Chondrocytes adapted for injection can also comprise, in certain aspects, chondrocytes which can be loosely connected or unattached to each other, and can be chondrocytes not comprised by cartilaginous tissue.

Certain embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1

This example illustrates procurement of chondrocytes from cadavers.

In this example, articular cartilage was obtained within 48-72 hours of death from local organ procurement organizations, including Mid-America Transplant Services (St. Louis, Mo.) and National Disease Research Interchange (Philadelphia, Pa.). Proper consent for inclusion in research was obtained from the next of kin. None of the donors received corticosteroids or cytostatic drugs as a treatment for arthritis. Visually intact articular cartilage and bone marrow was harvested from fifteen donors (both male and female) ranging in age from new-born to 48 years. “Knee-en-bloc” tissues were stored in DMEM at 4° C. pending serological reporting of possible viral contamination, and screened for contamination with microorganisms and viruses. The results typically were reported within 48-72 hours after submission. One case, an infant, required 5 days before the tissue was considered acceptable because no blood was drawn from the infant. In this case, additional sampling of maternal blood was required. This example illustrates that living chondrocytes can be obtained from cadavers.

### EXAMPLE 2

This example illustrates chondrocyte isolation.

In this example, articular chondrocytes were isolated and neocartilage disks were grown to Day 45-60 of culture as described previously (U.S. Pat. Nos. 6,235,316 and 6,645,764 to Adkisson; Adkisson et al., Clin. Orthop. Relat. Research. 391 Suppl., S280-294, 2001). Twenty-six neocartilage disks (two disks prepared from each of thirteen separate donors ranging in age from neonatal to 8 years) were digested overnight in HL-1 medium containing CLS4 collagenase (Worthington, Lakewood, N.J.) and hyaluronidase (type VIII, Sigma, St. Louis, Mo.). The dissociated chondrocytes were washed 2× in RPMI 1640 medium containing 2% FBS and resuspended in HL-1 medium. The cells were counted and diluted in RPMI containing 10% heat inactivated human AB serum, 10 mM HEPES, and 2 mM Glu and stored on ice until further use in either mixed lymphocyte reaction (MLR) assays or flow cytometric characterization. Final cell concentrations in these preparations were 1×10⁶ cells per ml.

### EXAMPLE 3

This example illustrates generation of bone marrow derived dendritic cells (BMDC).

In this example, bone marrow mononuclear cells served as a source of progenitor cells that were differentiated via in vitro manipulation to produce dendritic cells for use in mixed lymphocyte reactions (MLR). BMDC were used as a positive control in MLR assays for testing the immunoreactivity of chondrocytes that are isogeneic to the BMDC. In this procedure, the medullary space of the femur, tibia and fibula of each “knee-en-bloc” was flushed with Ca²⁺/Mg²⁺-free PBS to collect viable mononuclear cells on standard ficoll-histopaque (1.077 g/mL, Sigma, St. Louis, Mo.) within 96 h of death. Monocytes thereby obtained were washed 3× in Ca²⁺/Mg²⁺-free PBS and cryopreserved before expansion and differentiation in vitro to BMDC using a method adapted from Dubois et al., J. Immulogy 161, 2223-2231, 1998. Briefly, 3×10⁶ bone marrow-derived mononuclear cells were rapidly thawed at 37° C. and incubated in X-Vivo 15 serum-free medium (Cambrex, Walkersville, Md.) containing Flt-3 ligand (100 ng/mL), TNF-α (10 ng/mL), GM-SCF (100 ng/mL), IL-3 (10 ng/mL), IL-7 (10 ng/mL), SCF (10 ng/mL), 20 mM HEPES, 2 mM Glut and 5% human AB serum. On day 7 of culture, these cells were split 1:4 using trypsin/EDTA and maintained for 72 hrs in the marrow expansion medium identified above. On day 10 of culture, maturation of the dendritic precursor cells was stimulated by addition of IL-4 (20 ng/mL) to the same medium during the final 72 h of culture. Morphological characterization of BMDC revealed the presence of rounded cells that were loosely attached to the polystyrene culture surface. Phenotype and functional properties of these in vitro-generated BMDC were characterized by flow cytometry and a mixed lymphocyte reaction assay, described below.

### EXAMPLE 4

This example illustrates that chondrocytes do not stimulate a T cell response in a mixed lymphocyte reaction.

In this example, co-cultures of chondrocytes and allogeneic lymphocytes were established to assess the effector cell activity of neocartilage-derived chondrocytes. In these experiments, neocartilage chondrocytes (U.S. Pat. Nos. 6,235,316 and 6,645,764 to Adkisson; Adkisson et al., Clin. Orthop. Relat. Research. 391 Suppl., S280-294, 2001) and BMDC were obtained from the same donor. The BMDC were generated in vitro from aspirates that had been harvested from the tibial/femoral metaphyses at the time of cartilage procurement. These cells were subsequently expanded in vitro and differentiated under defined conditions for generating BMDC. The BMDC were found to be functionally active.

In these experiments, test stimulator cells, either chondrocytes or bone marrow derived dendritic cells (BMDC), were co-cultured in flat bottom plates at increasing concentration with 1×10⁵ allogeneic peripheral blood lymphocytes (PBL). Both the BMDC and the chondrocytes were γ-irradiated at 3000 rads, and served as stimulator cells in the mixed lymphocyte reactions. Non-irradiated peripheral blood lymphocytes (PBL) (1×10⁵) obtained from unrelated donors were used as the responder population in mixed lymphocyte reactions. In vitro proliferation of allogeneic lymphocytes was measured on day 7 of co-culture using stimulator cells of increasing concentration (10² to 10⁴ ) after an 18 h pulse with tritiated thymidine (Amersham, 1 μCi/mL, Piscataway, N.J.) in T cell media (RPMI containing 10% FBS, 15 mm HEPES, 2 mM L-Glutamine, 1 mM MEM Sodium Pyruvate Solution, 1× Sigma MEM Non-essential Amino Acid Solution, 1× Penicillin-Streptomycin (Gibco), 5×10⁻⁵ M mercaptoethanol and 8.9 mM sodium biacarbonate). Cells were lysed in water, and released DNA was bound to glass filters using an automated cell harvester. The filters were dried and counted in a Wallac MicroBeta Scintillation Counter (Perkin Elmer, Boston, Mass.).

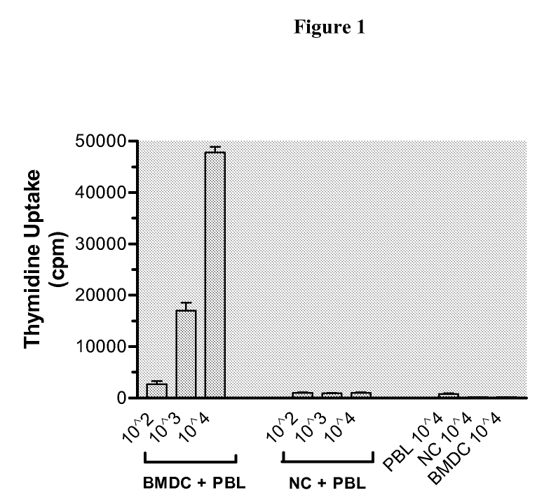

As shown in FIG. 1, the results indicate that BMDC were potent stimulators of alloreaction, but that chondrocytes harvested from the same donor tissue were incapable of stimulating a proliferative response in allogeneic T cells during in vitro co-culture. Results are expressed as the mean of six replicates ±SEM. Less than 500 counts were observed on average in control cultures of PBL, neocartilage (NC) or BMDC alone. These data indicate that chondrocytes are not immunostimulatory to allogeneic T cells.

### EXAMPLE 5

This example illustrates that chondrocytes down regulate immunological reactions.

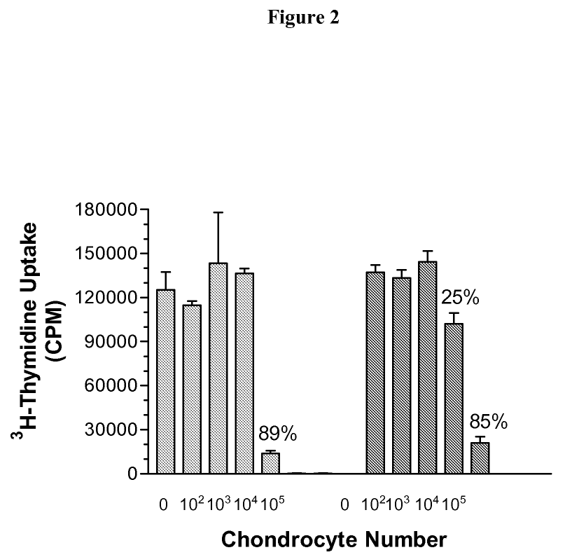

In this example, chondrocytes obtained as described above were subsequently co-cultured with activated T lymphocytes (FIG. 2). Purified CD4+T lymphocytes were obtained from the peripheral blood of normal human subjects by positive selection using magnetic-activated cell sorting separation (MACS) columns (Miltenyi Biotec, Auburn, Calif.). Naive T cells (105) were artificially activated in 96 well plates by applying crosslinking antibodies against both CD3 (10 ng/ml) and CD28 (5 ug/ml) (purchased from Pharmingen) at initiation of culture. In these experiments, allogeneic chondrocytes isolated from two separate donors were irradiated, then co-cultured with T cells in increasing concentration in culture wells each comprising 10⁵ CD4⁺ T cells. Lymphocyte proliferation was measured 5 days after activation via treatment with the crosslinking antibodies, and tritiated thymidine was added 16 h before harvest. Data represent the mean ±sd for tritiated thymidine uptake in quadruplicate samples. In these studies, CD4⁺ T cells showed tremendous proliferative potential within two to three days after activation. However, upon addition of chondrocytes, the T cells showed diminished proliferation in spite of crosslinking of the TCR and CD 28. The effect was dose dependent: addition of increasing numbers of chondrocytes resulted in a decrease in total radioactive counts such that up to 89% inhibition of lymphocyte proliferation was observed at a 1:1 ratio of chondrocytes to lymphocytes. These studies show that chondrocytes can down regulate T cell activation.

### EXAMPLE 6

This example illustrates that chondrocytes down regulate immunolgical reactions through direct cell-cell contact.

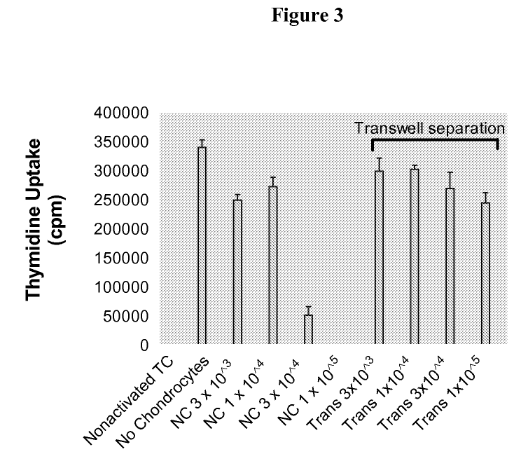

In this example, the assay system described in Example 5 was used to investigate if chondrocyte-mediated inhibition of lymphocyte proliferation resulted from diffusible factor(s) secreted by chondrocytes, or required direct cell-to-cell contact. In these experiments, chondrocytes were divided into two groups (FIG. 3). Group 1 chondrocytes were grown in direct contact with lymphocytes (left columns of FIG. 3), whereas chondrocytes from group 2 cultures were physically separated from direct contact with lymphocytes using Anapore Transwell Strips (Nunc, 0.2 micron pore size) which permit sharing of culture medium without direct contact between two cell populations (right columns of FIG. 3). Tritiated thymidine was added to each well during the final 18 hours of the 72 h incubation period, and incorporated radiolabel was measured by scintillation counting. As shown in FIG. 3, T-cell proliferation diminished with increasing amounts of chondrocytes when direct chondrocyte-to-lymphcyte contact was permitted. However, the inhibitory response was abolished by greater than 80% when cells were kept separated but shared the same medium.

These observations suggest that one or more cell surface molecules (and not a secreted paracrine-acting cytokine, such as TGF beta or IL-4) are responsible for the chondrocyte-mediated immunosuppressive effect observed in the MLR assay.

### EXAMPLE 7

This example illustrates flow cytometric analysis of cell surface antigens of chondrocytes.

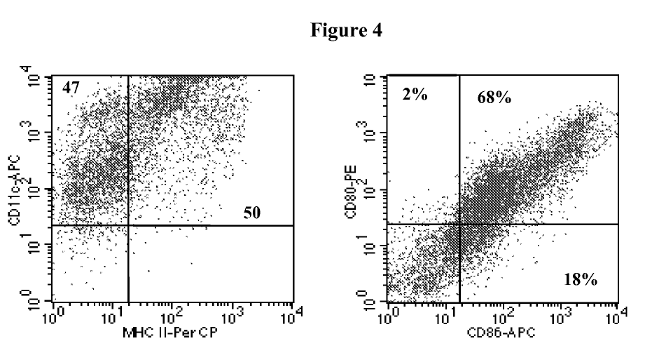

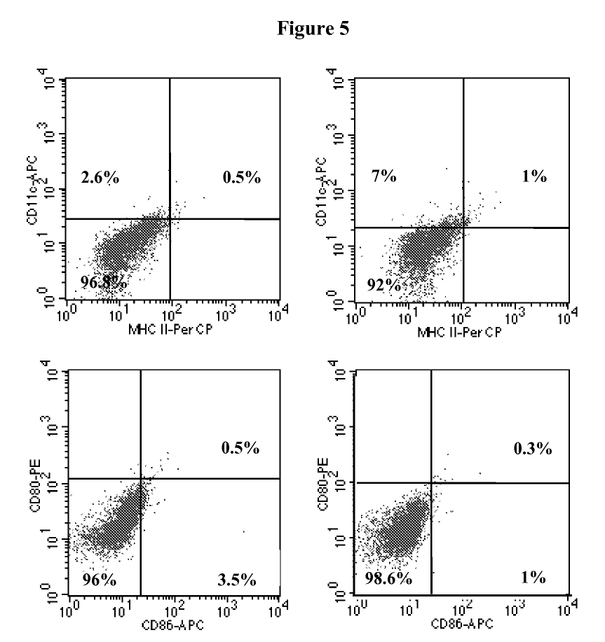

In this example, expression of cell surface markers commonly associated with effector cells were analyzed by flow cytometry in BMDC and chondrocytes (FIGS. 4 and 5). For these analyses, 150,000-200,000 cells were washed, and three-staining tubes were prepared for each sample. Cells were suspended in 100 μl of staining solution (DPBS with 2% FBS). The staining conditions were as follows: Tube 1—LIN, HLA-DR, CD11c, CD123; Tube 2—CD40, CD80, CD86; Tube 3—negative control. Data were analyzed using CellQuest software. To calculate the percentage of cells staining positive with antigen-specific monoclonal antibodies, the interface channel for positivity was set at 2% of the control fluorescence, using cells stained with isotype-matched control antibodies.

In these experiments, the flow cytometry analysis of BMDCs from a 3 month male revealed that these cells express cell surface markers CD11c, MHC II, CD80 and CD86 (FIG. 4). The data indicate that 50% of BMDC express both MHC II and CD11c, and that 68% of BMDCs express both CD80 and CD86 co-stimulatory cell surface markers. In contrast, as shown in FIG. 5, flow cytometry analysis of chondrocyte cell surface antigens using cells derived from two separate donors (a 5 week female, left column and a 3 month male, right column) demonstrated that these cells normally do not express CD11c, CD80 or CD86, whereas MHC class II essentially could not be identified in 98-99% of unstimulated juvenile chondrocytes. Subsequent studies showed that MHC class II alone, and not CD80 or CD86 were induced on the chondrocyte cell surface following treatment with inflammatory cytokines such as IFN-γ and TNF-α (data not shown). These data indicate that chondrocytes do not express at least three cell surface markers expressed by professional antigen presenting cells.

### EXAMPLE 8

This example describes a comparative assessment of chondrocyte and BMDC immunogenicity.

In these experiments, lymphocyte proliferation was measured in mixed cultures of peripheral blood lymphocytes and chondrocytes or peripheral blood lymphocytes and BMDCs. As shown in the table, BMDCs consistently stimulated lymphocyte proliferation, but chondrocytes did not.

| | | | Chondrocyte | BMDC |
|---|---|---|---|---|
| Donor | Age | | MLR | MLR* |
| Female | 5 | wks. | Backgnd. | +++ |
| Male | 6 | wks. | Backgnd. | NA |
| Male | 2 | mo. | Backgnd. | ++ |
| Male | 3 | mo. | Backgnd. | ++ |
| Female | 3 | mo. | Backgnd. | ++ |
| Male | 3 | mo. | Backgnd. | + |
| Male | 3 | mo. | Backgnd. | ++ |
| Female | 4 | mo. | Backgnd. | +++ |
| Female | 1.2 | yrs. | + | +++ |
| Male | 2.6 | yrs. | Backgnd. | +++ |
| Male | 2.75 | yrs. | Backgnd. | +++ |
| Female | 5 | yrs. | Backgnd. | +++ |
| Male | 6 | yrs. | Backgnd. | NA |
| Female | 8 | yrs. | Backgnd. | NA |

*+ = <10-fold stimulation at 10⁴ cells
++ = 20-40-fold stimulation at 10⁴ cells
+++ = >50-fold stimulation at 10⁴ cells

### EXAMPLE 9

This example illustrates changes in gene expression in aging chondrocytes.

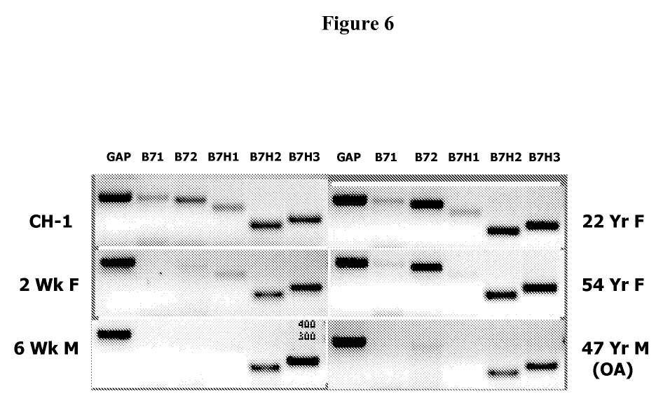

In this example, reverse transcription combined with polymerase chain reaction (RT-PCR) was used to analyze mRNA expression in chondrocytes. In this example, mRNA levels for various genes were measured semi-quantitatively using RT-PCR in chondrosarcoma cells and chondrocytes obtained from donors of various ages. As shown in FIG. 6, a chondrosarcoma cell line, CH-1, expressed markers GAP (control), B71, B72, B7H2 and B7H3. In contrast, gene expression in chondrocytes from various human sources was highly variable, particularly regarding expression of B71, B72, and B7H1. Note, for example, background levels of expression of these markers in young donors (2 week female and 6 week male), and in a 47 yr osteoarthritic male, and the high level of expression of B72 in adults (22 yr and 54 yr females). However, consistent expression of B7H2 and B7H3 was observed in all samples tested, suggesting that these markers, either alone or in combination, and possibly Interferon-γ-inducible expression of B7H 1, can provide a signal that is necessary and sufficient to block proliferation of CD4+ T cells.

### EXAMPLE 10

This example illustrates intra-articular delivery of allogeneic chondrocytes in sodium hyaluronate carrier for the repair of cartilagenous joint structures in a model mammalian system. Two different models ofjoint disease can be investigated in this system. The first model focuses on isolated lesions created in the weight bearing region of the femoral condyle to simulate traumatic knee injury, while the second model involves transection of the medial meniscus to create a mechanically unstable knee. The latter model was developed originally to simulate degenerative changes commonly found in osteoarthritic joints (Ghosh, P., et al., Clin. Orthop. Rel. Res. 252, 101-113, 1990; Ghosh, P., et al., Sem. Arth. Rheum. 22 Suppl. 1, 18-30, 1993; Ghosh, P.,et al., Sem. Arth. Rheum. 22 Suppl. 1, 31-42, 1993; Hope N, et al., Sem. Arth. Rheum. 22 Suppl. 1, 43-51, 1993.)

In this system, sheep matched for gender, size and age (e.g., thirty 2-4 year old ewes) can be equally divided to assess cartilage repair after intra-articular delivery of juvenile ovine chondrocytes in sodium hyaluronate carrier. Six of the thirty animals can be kept as unoperated controls. Sheep of similar body weight (45-80 kg) can be purchased from a single supplier. Twelve of these animals can be randomly selected for inclusion in the first arm of the study in which full-thickness defects can be created in the medial femoral condyle. Surgery can be carried out under general anesthesia, using halothane and oxygen inhalation via endotracheal tubing. Medial stifle arthrotomies can be performed on the right hind limb of each sheep, exposing the medial femoral condyle. A circular defect (5-8 mm diameter, 500-700 um deep) can be created in the weight bearing region of the medial femoral condyle using a custom designed stainless steel punch and a #15 scalpel blade without violating subchondral bone. Arthrotomies can be closed in layers using absorbable suture materials. Upon recovery from anesthesia, these animals can be returned to their holding pens. Administration of vehicle, and chondrocytes plus vehicle can be as described in detail below. Animals used in each arm of the study can be administered prophylactic antibiotics prior to recovery from anesthesia, and analgesics can be given twice daily for a period of three days. After a two week recovery period, all sheep can be exercised 5 days per week for a period of 12 weeks to induce degenerative changes in the knee. Exercise can consist of walking the sheep in a run of approximately 100 m in length per day.

Animals designated for the second arm of the study can be operated on as described below, Briefly, unilateral medial meniscectomy can be performed on the right knee of 12 animals using the surgical procedure described by Ghosh, P., et al., Clin. Orthop. Rel. Res. 252, 101-113. Twelve weeks after meniscectomy, sheep in each arm of the study (full-thickness defect model and meniscectomy model) can be randomly divided into two treatment groups of six animals each. Group 1 animals can receive a single injection (5 mL) of sterile sodium hyaluronate with a molecular mass of 2 million daltons (4 mg/mL in saline, Hyaluron, Inc. Burlington, Mass.). Group 2 animals can receive the same preparation in which chondrocytes are resuspended at a concentration of 1 million per ml. Chondrocytes derived from ovine articular cartilage (male, new-born to 6 months of age) can be first expanded in chemically defined, serum-free medium containing cytokines and ascorbate, according to U.S. patent application Ser. No. 10/956,971 to Adkisson et al. These cells can be cryopreserved after expansion at 10 million per mL using Cryostore Solution (BioLife Solutions, Binghamton, N.Y.). Immediately prior to use, individual vials can be rapidly thawed at 37° C. and washed in HL-1 Complete Serum-Free Medium (Cambrex, Walkersville, Md.). Cell pellets can be combined after washing for resuspension in sodium hylauronate as described above. Viability of freshly thawed chondrocytes after washing can be 85-97% as measured using a Gauva Personal Cell Analysis System and fluorescent reagents purchased from Guava Technologies, Inc. (Hayward, Calif.).

Sheep can be weighed before treatment, and blood can be collected to obtain serum for analysis. The stifle joint can be shaved and the animal prepared for anesthesia. Radiographs can be obtained of both knees. Once the animals are intubated and placed on the table in the dorsal recumbency position, the right stifle can be prepared for surgery using iodine and Hibiclens (Zeneca), and rinsed with sterile water. The joint can be flexed 20 times to circulate synovial fluid. The stifle can be placed at 70-90 degrees of flexion to remove as much synovial fluid as possible in order to make room for injection of the treatment regimen (sodium hyaluronate alone or sodium hyaluronate+chondrocytes). With the knee in the same position, 10-20 mL of sterile saline can be introduced into the joint space to remove all traces of synovial fluid. An 18 G needle can be inserted proximal to the meniscal/tibial plateau and the notch formed by their junction. After flexing and extending the joint 20 times, fluid can be aspirated from the joint. A three-way stopcock with an 18 G needle attached can be inserted into the triangle described above on the medial side of the joint, just medial to the patellar ligament. A syringe containing the chondrocyte suspension described above can be attached to the stopcock. Once attached, the stopcock can be opened and the cell suspension injected slowly into the joint space. Residual preparation material remaining in the syringe can be washed with 1 mL volume of sterile saline. The stifle can be flexed and extended 20 times to distribute the chondrocyte suspension in the joint space, and the sheep can then be maintained in the recumbency position for a minimum of 10 minutes before recovery and transfer to a holding pen.

At 16 weeks post-injection, sheep can be sacrificed by overdose with Euthasol (sodium pentobarbital, 100-200 mg/kg, IV). Lymph nodes draining the joint can then be obtained from the operated and contralateral limb for comparison to unoperated control animals after obtaining wet weights. The leg can then be disarticulated at the hip, and radiographs of the stifle obtained. Synovial fluid can be collected using an 18 G needle. 10 mL of sterile saline can be injected into the joint space and the lavage fluid collected and saved for analysis. The stifle can be dissected and the gross morphological appearance of all joint structures, including the presence or absence of osteophytes, can be documented with digital photography. The following tissues can be collected for histological examination of chondrocyte attachment and cartilage repair: lymph nodes, synovial capsule, fat pad, posterior and anterior cruciate ligaments and both native and repair meniscal tissue. After dissection, 13 areas of cartilage on both the operated and contralateral control joints and both joints of the unoperated control animals can be graded visually using the criteria described by Murphy et al., Arthritis & Rhematism 48, 3464-3474 2003.

The selected areas can be located on the protected and unprotected regions of the medial and lateral tibial plateaus, the anterior, middle and posterior aspect of the medial condyle, the middle and posterior regions of the lateral condyle, the lateral, central and medial regions of the trochlear ridge and on the patella. Tissue sections can be collected using a band saw and immediately fixed in 10% neutral buffered formalin for histological characterization of cartilage and bone structure using safraninO/fast green, as well as pentachrome after decalcification in 14% EDTA. Tracking of male chondrocytes to various joint tissues can be determined by fluorescence in situ hybridization using a Y chromosome-specific probe.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above may include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

All publications, patents, patent applications and other references cited in this application are herein incorporated by reference in their entirety as if each individual publication, patent, patent application or other reference were specifically and individually indicated to be incorporated by reference.

What is claimed is:

1. A method of treating joint disease in a mammal in need thereof, the method comprising:
forming a composition comprising chondrocytes expressing type II collagen and at least one biological macromolecule; and
injecting the composition into a diseased joint in the mammal.

2. A method in accordance with claim 1, further comprising growing the chondrocytes expressing type II collagen in vitro.

3. A method in accordance with claim 1, wherein the mammal is a human patient in need of treatment and wherein the chondrocytes are human chondrocytes.

4. A method in accordance with claim 1, wherein the joint disease is osteoarthritis.

5. A method in accordance with claim 4, wherein the osteoarthritis comprises degenerative articular cartilage.

6. A method in accordance with claim 4, wherein the osteoarthritis comprises a degenerative meniscus.

7. A method in accordance with claim 1, wherein the diseased joint is a joint other than an intervertebral disc.

8. A method in accordance with claim 1, wherein the diseased joint is a joint selected from the group consisting of a knee joint, a hip joint, a shoulder joint, an ankle joint, a wrist joint, a digit joint and an elbow joint.

9. A method in accordance with claim 1, wherein the diseased joint is a joint selected from the group consisting of a knee joint, a hip joint, and a shoulder joint.

10. A method in accordance with claim 1, wherein the chondrocytes expressing type II collagen are chondrocytes expressing high molecular weight sulfated proteoglycan.

11. A method in accordance with claim 1, wherein the chondrocytes expressing type II collagen are cadaver chondrocytes expressing type II collagen.

12. A method in accordance with claim 11, wherein the cadaver chondrocytes expressing type II collagen are hyaline cartilage cadaver chondrocytes expressing type II collagen.

13. A method in accordance with claim 1, further comprising isolating the chondrocytes from a cadaver deceased for up to about fourteen days at time of the isolating.

14. A method in accordance with claim 1, further comprising isolating the chondrocytes from a cadaver no older than about fourteen years of age at time of death.

15. A method in accordance with claim 1, wherein the at least one biological macromolecule is selected from the group consisting of hyaluronic acid, type I collagen, type III collagen, fibrinogen, fibrin, thrombin, pectin and chitosan.

16. A method in accordance with claim 1, wherein the at least one biological macromolecule is hyaluronic acid, wherein said hyaluronic acid has an average molecular mass of at least about 1×10⁶ daltons.

17. A method in accordance with claim 1, wherein the at least one biological macromolecule is selected a collagen from the group consisting of type I collagen, type III collagen, and a combination thereof.

18. An apparatus configured for injection of chondrocytes expressing type II collagen into a diseased non-intervertebral joint of a mammal, the apparatus comprising:
a) a reservoir holding therewithin a composition comprising at least one biological macromolecule and chondrocytes expressing type II collagen; and
b) at least one hollow tube which inserts into the diseased non-intervertebral joint, wherein the hollow tube is communicably connected with the reservoir.

19. An apparatus in accordance with claim 18, wherein the mammal is a human patient in need of treatment and wherein the chondrocytes are human chondrocytes.

20. An apparatus in accordance with claim 18, wherein the chondrocytes expressing type II collagen are chondrocytes from a cadaver no older than about 14 years of age at time of death.

21. An apparatus in accordance with claim 18, wherein the chondrocytes expressing type II collagen are human cadaver chondrocytes isolated from a cadaver deceased for up to about fourteen days at the time of the isolating.

22. An apparatus in accordance with claim 21, wherein the human cadaver chondrocytes isolated from a cadaver deceased for up to about fourteen days at the time of the isolating are chondrocytes grown in vitro.

23. An apparatus in accordance with claim 18, wherein the diseased non-intervertebral joint of a mammal is selected from the group consisting of a knee joint, a hip joint, a shoulder joint, an ankle joint, a wrist joint, a digit joint and an elbow joint.

24. An apparatus in accordance with claim 18, wherein the chondrocytes expressing type II collagen are chondrocytes expressing high molecular weight sulfated proteoglycan.

25. An apparatus in accordance with claim 18, wherein the at least one biological macromolecule is selected from the group consisting of hyaluronic acid, type I collagen, type III collagen, fibrinogen, fibrin, thrombin, pectin and chitosan.

26. An apparatus in accordance with claim 18, wherein the at least one biological macromolecule is hyaluronic acid, wherein said hyaluronic acid has an average molecular mass of at least about 1×10⁶ daltons.

27. An apparatus in accordance with claim 18, wherein the at least one biological macromolecule is a collagen selected from the group consisting of type I collagen, type III collagen and a combination thereof.

28. An apparatus in accordance with claim 18, wherein the hollow tube is hollow needle.
